(19) United States
(12) Patent Application Publication
(10) Pub. No.: US 2025/0331863 A1
(43) Pub. Date: Oct. 30, 2025

(54) SYSTEMS FOR OCCLUDING A LEFT ATRIAL APPENDAGE

(71) Applicant: BOSTON SCIENTIFIC SCIMED, INC., MAPLE GROVE, MN (US)

(72) Inventors: Patrick Willoughby, Shoreview, MN (US); Scott Andrew McCullough, Plymouth, MN (US)

(73) Assignee: BOSTON SCIENTIFIC SCIMED, INC., MAPLE GROVE, MN (US)

(21) Appl. No.: 19/187,052

(22) Filed: Apr. 23, 2025

Related U.S. Application Data
(60) Provisional application No. 63/638,011, filed on Apr. 24, 2024.

(51) Int. Cl. A61B 17/12; A61B 17/00

(52) U.S. Cl. CPC A61B 17/12122; A61B 17/12181; A61B 2017/00867

(57) ABSTRACT

A left atrial appendage closure (LAAC) device is adapted for occluding a patient's left atrial appendage (LAA). The LAAC device includes a shape memory foam occlusive element that is adapted to expand from a crimped configuration for delivery to an expanded configuration after deployment, the shape memory foam occlusive element including in its expanded configuration a distal face, a proximal face and an intervening periphery. A reinforcing member is disposed relative to the shape memory foam occlusive element and is adapted to limit deformation of the shape memory foam occlusive element after the shape memory foam occlusive element has expanded into the expanded configuration.

### CROSS-REFERENCE TO RELATED APPLICATION

This application is a continuation of U.S. Patent Application Ser. No. 63/638,011, filed Apr. 24, 2024, entitled “SYSTEMS FOR OCCLUDING A LEFT ATRIAL APPENDAGE”, which is incorporated by reference herein in its entirety.

### TECHNICAL FIELD

The disclosure relates generally to medical devices and more particularly to systems for occluding a left atrial appendage.

### BACKGROUND

Medical devices implanted within the heart may include left atrial appendage closure (LAAC) devices, which are intended to close off the left atrial appendage (LAA) in order to reduce the likelihood of thrombi forming in the LAA from escaping the LAA and entering the bloodstream. Thrombi that migrate through the blood vessels may eventually plug a smaller vessel downstream and thereby contribute to stroke or heart attack. Clinical studies have shown that the majority of blood clots in patients with atrial fibrillation originate in the LAA. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a left atrial appendage closure (LAAC) device that is adapted for occluding a patient's left atrial appendage (LAA). The LAAC device includes a shape memory foam occlusive element that is adapted to expand from a crimped configuration for delivery to an expanded configuration after delivery. The shape memory foam occlusive element includes when in its expanded configuration a distal face, a proximal face and an intervening periphery. A reinforcing member is disposed relative to the shape memory foam occlusive element and is adapted to limit deformation of the shape memory foam occlusive element after the shape memory foam occlusive element has expanded into the expanded configuration.

Alternatively or additionally, the reinforcing member may extend around at least a portion of the periphery of the shape memory foam occlusive element.

Alternatively or additionally, the reinforcing member may extend across at least a portion of the distal face of the shape memory foam occlusive element.

Alternatively or additionally, the reinforcing member may extend across at least a portion of the proximal face of the shape memory foam occlusive element.

Alternatively or additionally, at least a portion of the reinforcing member may extend through the shape memory foam occlusive element.

Alternatively or additionally, the reinforcing member may be adapted to expand with the shape memory foam occlusive element when the shape memory foam occlusive element expands after deployment.

Alternatively or additionally, the reinforcing member may include a second foam different from a first foam forming the shape memory foam occlusive element.

Alternatively or additionally, the reinforcing member may include a polymeric structure.

Alternatively or additionally, the reinforcing member may include a metallic structure.

Another example may be found in a left atrial appendage closure (LAAC) device that is adapted for occluding a patient's left atrial appendage (LAA) that includes a proximal mouth. The LAAC device includes a deformable device body that is adapted to expand and span the proximal mouth of the LAA and a control structure that is adapted to limit deformation of the deformable device body once expanded.

Alternatively or additionally, the deformable device body may include a shape memory foam.

Alternatively or additionally, the control structure may extend around at least a portion of a periphery of the deformable device body.

Alternatively or additionally, the control structure may extend across at least a portion of a distal side of the deformable device body.

Alternatively or additionally, the reinforcing member may extend across at least a portion of a proximal side of the deformable device body.

Alternatively or additionally, at least a portion of the reinforcing member may extend through the deformable device body.

Another example may be found in a left atrial appendage closure (LAAC) device that is adapted for occluding a patient's left atrial appendage (LAA). The LAAC device includes a shape memory foam occlusive element that is adapted to expand from a delivery configuration to a deployment configuration, and a reinforcing member that is disposed relative to the shape memory foam occlusive element and that includes a central point and a plurality of struts emanating outwardly from the central point.

Alternatively or additionally, the reinforcing member may be adapted to limit deformation of the shape memory foam occlusive element after the shape memory foam occlusive element has expanded.

Alternatively or additionally, the reinforcing member may further include an annular ring extending around and connected to at least some of the plurality of struts.

Alternatively or additionally, the annular ring has a diameter that may be equal to or less than an overall diameter of the shape memory foam occlusive element.

Alternatively or additionally, the reinforcing member may further include one or more foam integration structures emanating from at least some of the plurality of struts.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional view of a left atrial appendage (LAA) with an illustrative left atrial appendage closure (LAAC) device deployed therein;

FIG. 2 is a schematic side view of an illustrative LAAC device;

FIG. 3 is a schematic side view of an illustrative LAAC device;

FIG. 4 is a schematic side view of an illustrative LAAC device shown in an expanded configuration;

FIG. 5 is a schematic side view of the illustrative LAAC device of FIG. 4, shown in a compressed configuration;

FIG. 6 is a schematic end view of an illustrative LAAC device;

FIG. 7 is a schematic end view of an illustrative LAAC device;

FIG. 8 is a schematic end view of an illustrative LAAC device;

FIG. 9 is a schematic end view of an illustrative LAAC device;

FIG. 10 is a schematic end view of an illustrative LAAC device;

FIG. 11 is a schematic end view of an illustrative LAAC device;

FIG. 12 is a schematic end view of an illustrative LAAC device;

FIG. 13 is a schematic end view of an illustrative LAAC device;

FIG. 14 is a schematic end view of an illustrative LAAC device;

FIG. 15 is a schematic end view of an illustrative LAAC device;

FIG. 16 is a schematic end view of an illustrative LAAC device; and

FIG. 17 is a schematic end view of an illustrative LAAC device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

A left atrial appendage closure (LAAC) device may be adapted for occluding a patient's left atrial appendage (LAA). The LAAC device includes a shape memory foam occlusive element that is adapted to expand from a crimped configuration for delivery to an expanded configuration after delivery. The shape memory foam occlusive element includes, when in its expanded configuration a distal face, a proximal face and an intervening periphery. A reinforcing member is disposed relative to the shape memory foam occlusive element and is adapted to limit deformation of the shape memory foam occlusive element after the shape memory foam occlusive element has expanded into the expanded configuration that could otherwise result in axial movement of the LAAC device. In some cases, the reinforcing member may help to hold the shape memory foam occlusive element in place within the LAA by limiting the deformation or bending of the shape memory foam occlusive element in response to applied forces such as the forces applied by the beating heart, and by blood flowing either in or out of the LAA.

In some cases, the reinforcing member may extend around at least a portion of the periphery of the shape memory foam occlusive element. In some cases, the reinforcing member may extend across at least a portion of the distal face of the shape memory foam occlusive element. In some cases, the reinforcing member may extend across at least a portion of the proximal face of the shape memory foam occlusive element. In some cases, at least a portion of the reinforcing member may extend through the shape memory foam occlusive element. The reinforcing member may be adapted to expand with the shape memory foam occlusive element when the shape memory foam occlusive element expands after deployment. As an example, the reinforcing member may include a second foam different from a first foam forming the shape memory foam occlusive element. As an example, the reinforcing member may include a polymeric structure. As an example, the reinforcing member may include a metallic structure.

A left atrial appendage closure (LAAC) device that is adapted for occluding a patient's left atrial appendage (LAA) includes a deformable device body that is adapted to expand and span a proximal mouth of the LAA and a control structure that is adapted to limit deformation of the deformable device body once expanded. In some cases, the deformable device body may include a shape memory foam. In some cases, the control structure may extend around at least a portion of a periphery of the deformable device body. In some cases, the control structure may extend across at least a portion of a distal side of the deformable device body. In some cases, the reinforcing member may extend across at least a portion of a proximal side of the deformable device body. In some cases, at least a portion of the reinforcing member may extend through the deformable device body.

A left atrial appendage closure (LAAC) device adapted for occluding a patient's left atrial appendage (LAA) includes a shape memory foam occlusive element that is adapted to expand from a delivery configuration to a deployment configuration and a reinforcing member that is disposed relative to the shape memory foam occlusive element. The reinforcing member includes a central point and a plurality of struts emanating outwardly from the central point. In some cases, the reinforcing member may be adapted to limit deformation of the shape memory foam occlusive element after the shape memory foam occlusive element has expanded. In some cases, the reinforcing member may further include an annular ring extending around and connected to at least some of the plurality of struts. As an example, the annular ring may have a diameter that is equal to or less than an overall diameter of the shape memory foam occlusive element. In some cases, the reinforcing member may further include one or more foam integration structures that emanate from at least some of the plurality of struts.

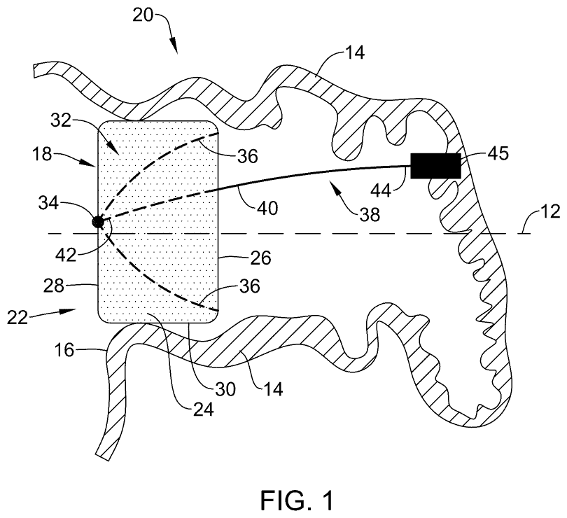

FIG. 1 is a partial cross-sectional view of a left atrial appendage 10. In some embodiments, the left atrial appendage (LAA) 10 may have a complex geometry and/or irregular surface area. It will be appreciated that the illustrated LAA 10 is merely one of many possible shapes and sizes for the LAA 10, which may vary from patient to patient. Those of skill in the art will also recognize that the medical devices, systems, and/or methods disclosed herein may be adapted for various sizes and shapes of the LAA 10, as necessary. The left atrial appendage 10 may include a generally longitudinal axis 12 arranged along a depth of a main body 20 of the left atrial appendage 10. The main body 20 may include a lateral wall 14 and an ostium 16 forming a proximal mouth 18. In some examples, a lateral extent of the ostium 16 and/or the lateral wall 14 may be smaller or less than a depth of the main body 20 along the longitudinal axis 12, or a depth of the main body 20 may be greater than a lateral extent of the ostium 16 and/or the lateral wall 14. In some examples, the LAA 10 may narrow quickly along the depth of the main body 20 or the left atrial appendage may maintain a generally constant lateral extent along a majority of depth of the main body 20. In some examples, the LAA 10 may include a distalmost region formed or arranged as a tail-like element associated with a distal portion of the main body 20. In some examples, the distalmost region may protrude radially or laterally away from the longitudinal axis 12.

As shown, an LAAC device 22 has been disposed within the LAA 10 such that the LAAC device 22 spans across the proximal mouth 18 of the LAA 10. In some cases, the LAAC device 22 may include a deformable device body 24. The deformable device body 24 may be considered as being a shape memory foam occlusive element that is adapted to expand from a crimped configuration for delivery to an expanded configuration after delivery (as shown). In some cases, the shape memory foam occlusive element, when in its expanded configuration, may be considered as including a distal face 26 that is adapted to face distally, a proximal face 28 that is adapted to face proximally, and an intervening periphery 30 that extends between the distal face 26 and the proximal face 28. In some cases, the periphery 30 may be considered as forming a side of the deformable device body or shape memory foam occlusive element 24. The LAAC device 22 includes a reinforcing member 32 that is disposed relative to the shape memory foam occlusive element 24 and is adapted to limit deformation of the shape memory foam occlusive element 24 after the shape memory foam occlusive element 24 has expanded into the expanded configuration. In some cases, the reinforcing member 32 may be considered as being a control structure.

In some cases, the reinforcing member 32 may extend around at least a portion of the periphery 30 of the shape memory foam occlusive element 24. In some cases, the reinforcing member 32 may extend across at least a portion of the distal face 26 of the shape memory foam occlusive element 24. In some cases, the reinforcing member 32 may extend across at least a portion of the proximal face 28 of the shape memory foam occlusive element 24. At least a portion of the reinforcing member 32 may extend through the shape memory foam occlusive element 24. In some cases, the reinforcing member 32 may be adapted to expand with the shape memory foam occlusive element 24 when the shape memory foam occlusive element expands after deployment 24. In some cases, the reinforcing member 32 may include a second foam different from a first foam forming the shape memory foam occlusive element 24. For example, the reinforcing member 32 may be formed of a foam having a different formulation, a different porosity or a different reticulation relative to the foam forming the shape memory foam occlusive element 24. The reinforcing member 32 may be formed of a shape memory polymer or a shape memory foam, for example. In some cases, the reinforcing member 32 may be a polymeric structure, a metallic structure, or may combine both polymer and metal.

As shown in FIG. 1, the reinforcing member 32 includes a central point 34 that is positioned near the proximal face 28 and several struts 36 that emanate distally from the central point 34. While FIG. 1 shows the reinforcing member 32 as including two struts 36, the reinforcing member 32 may include any number of struts 36. In some cases, the central point 34 may extend proximally from the proximal face 28 of the shape memory foam occlusive element 24, and may serve as an attachment point for temporarily attaching the LAAC device 22 to a delivery device (not shown). In some cases, the LAAC device 22 may be delivered without utilizing the central point 34 to grasp the LAAC device 22. In such cases, the central point 34 may not be exposed on the proximal face 28 of the shape memory foam occlusive element 24, and instead may be disposed within the shape memory foam occlusive element 24. In some cases, the central point 34 may be a weld joint between ends of each of the struts 36.

In some cases, the LAAC device 22 may include an anchor assembly 38 that allows the LAAC device 22 to be anchored within the LAA 10. As an example, the anchor assembly 38 may include an elongate member 40 extending from a proximal end 42 that is secured to the central point 34 to a distal end 44. In some cases, the distal end 44 may include an anchor 45. The anchor 45 may be any of a variety of different types of anchors. In some cases, the anchor assembly 38 may not be attached to the central point 34, and may instead extend distally from one of the struts 36. In some instances, the LAAC device 22 may not include the anchor assembly 38.

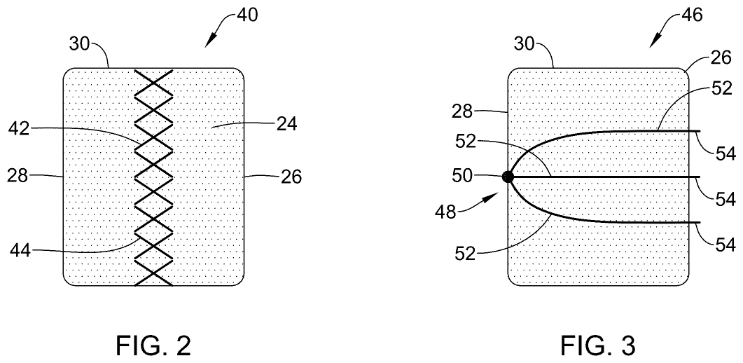

The reinforcing member 32 may take a variety of different forms. FIGS. 2 through 17 provide illustrative but non-limiting examples of reinforcing members that may be utilized in combination with the shape memory foam occlusive element 24 in forming an LAAC device. It will be appreciated that while the Figures show a number of permutations for the reinforcing member, in some cases an LAAC device may have a reinforcing member that includes combinations of particular features shown in various Figures.

FIG. 2 is a schematic side view of an illustrative LAAC device 40 that combines the shape memory foam occlusive element 24 with a reinforcing member 42. The reinforcing member 42 may be considered as being an example of the reinforcing member 32 shown in FIG. 1. As shown, the reinforcing member 42 extends around the periphery 30 of the shape memory foam occlusive element 24. In some cases, the reinforcing member 42 may be adapted to collapse down to accommodate a compressed or crimped delivery configuration for the shape memory foam occlusive element 24, and may be adapted to automatically expand into an expanded configuration when the LAAC device 40 is delivered and is allowed to expand within the LAA 10. The reinforcing member 42 may be a polymeric or metallic structure that includes a number of struts 44 that interact with each other in a way that allows the reinforcing member 42 to move between a collapsed configuration and an expanded configuration.

FIG. 3 is a schematic side view of an illustrative LAAC device 46 that combines the shape memory foam occlusive element 24 with a reinforcing member 48. The reinforcing member 48 may be considered as being an example of the reinforcing member 32 shown in FIG. 1. As shown, the reinforcing member 48 includes a central point 50 that is shown as being visible along the proximal face 28 of the shape memory foam occlusive element 24. In some cases, the central point 50 may be disposed within the shape memory foam occlusive element 24, distal of the proximal face 28. The reinforcing member 48 includes several struts 52 that extend distally from the central point 50, and extend through the shape memory foam occlusive element 24. In some cases, as shown, each of the struts 52 may include distal ends 54 that extend distally of the distal face 26. In some cases, at least some of the distal ends 54 of the struts 52 may terminate proximally of the distal face 26. While a total of three struts 52 are shown, this is merely illustrative, as the reinforcing member 48 may include any number of struts 52. In some cases, the reinforcing member 48 may be adapted to collapse down to accommodate a compressed or crimped delivery configuration for the shape memory foam occlusive element 24, and may be adapted to automatically expand into an expanded configuration when the LAAC device 46 is delivered and is allowed to expand within the LAA 10.

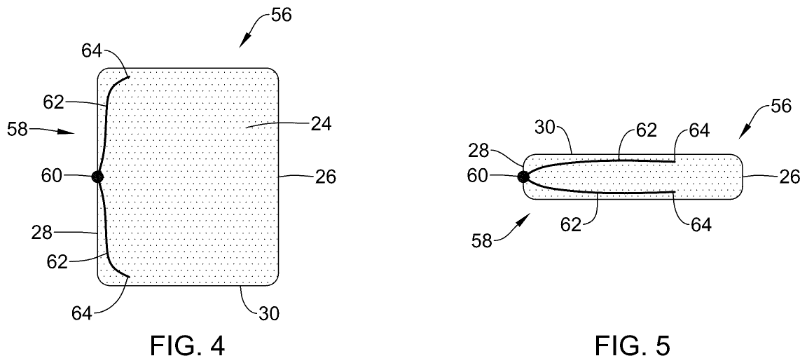

FIG. 4 is a schematic side view of an illustrative LAAC device 56 shown in an expanded configuration while FIG. 5 shows the LAAC device 56 in a collapsed or compressed configuration. The LAAC device 56 combines the shape memory foam occlusive element 24 with a reinforcing member 58. The reinforcing member 58 may be considered as being an example of the reinforcing member 32 shown in FIG. 1. As shown, the reinforcing member 58 includes a central point 60 that is shown as being visible along the proximal face 28 of the shape memory foam occlusive element 24. In some cases, the central point 60 may be disposed within the shape memory foam occlusive element 24, distal of the proximal face 28. The reinforcing member 58 includes several struts 62 that extend distally from the central point 50, and extend into the shape memory foam occlusive element 24. In some cases, as shown, each of the struts 62 may include distal ends 64 that terminate within the shape memory foam occlusive element 24 and thus do not extend to or through the distal face 26 of the shape memory foam occlusive element 24. In some cases, at least some of the struts 62 may have distal ends 64 that reach or even extend through the distal face 26. While a total of two struts 62 are shown, this is merely illustrative, as the reinforcing member 58 may include any number of struts 62. In some cases, the reinforcing member 58 may be adapted to collapse down to accommodate a compressed or crimped delivery configuration for the shape memory foam occlusive element 24, and may be adapted to automatically expand into an expanded configuration when the LAAC device 56 is delivered and is allowed to expand within the LAA 10.

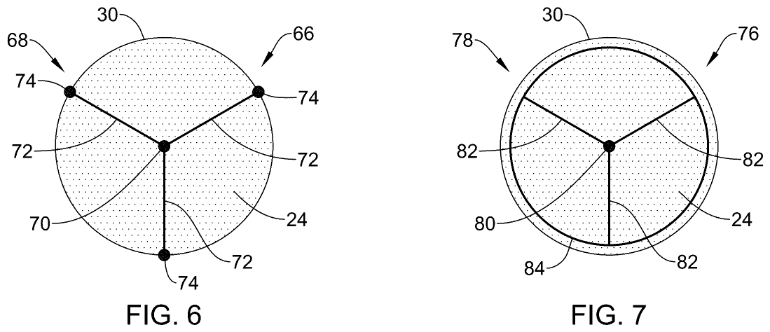

FIG. 6 is a schematic end view of an illustrative LAAC device 66 that combines the shape memory foam occlusive element 24 with a reinforcing member 68. The reinforcing member 68 may be considered as being an example of the reinforcing member 32 shown in FIG. 1. While a circular shape of the LAAC device 66 is shown, this is merely illustrative, as the LAAC device 66 may assume other shapes. As shown, the reinforcing member 68 includes a central point 70 that may or may not be visible along the proximal face 28. The reinforcing member 68 includes several struts 72 that extend radially outwardly from the central point 70, and extend axially into the shape memory foam occlusive element 24. In some cases, each of the struts 72 terminate in a ball 74. In some cases, the ball 74 for each strut 72 may be positioned at or near the periphery 30 of the shape memory foam occlusive element 24. In some cases, the ball 74 for at least some of the struts 72 may be positioned within the shape memory foam occlusive element 24, and may not be visible. While three struts 72 are shown, this is merely illustrative, as the reinforcing member 68 may include any number of struts 72. The balls 74 may be considered as examples of foam integration structures that help to anchor the reinforcing member 68 within the shape memory foam occlusive element 24. In some cases, one or more of the struts 72 may simply terminate, without a ball 74. In some cases, the reinforcing member 68 may be adapted to collapse down to accommodate a compressed or crimped delivery configuration for the shape memory foam occlusive element 24, and may be adapted to automatically expand into an expanded configuration when the LAAC device 66 is delivered and is allowed to expand within the LAA 10.

FIG. 7 is a schematic end view of an illustrative LAAC device 76 that combines the shape memory foam occlusive element 24 with a reinforcing member 78. The reinforcing member 78 may be considered as being an example of the reinforcing member 32 shown in FIG. 1. While a circular shape of the LAAC device 76 is shown, this is merely illustrative, as the LAAC device 76 may assume other shapes. As shown, the reinforcing member 78 includes a central point 80 that may or may not be visible from along the proximal face 28. The reinforcing member 78 includes several struts 82 that extend radially outwardly from the central point 80, and extend axially into the shape memory foam occlusive element 24. In some cases, as shown, each of the struts 82 terminate at an outer ring 84. In some cases, the outer ring 84 may be positioned at or near the periphery 30 of the shape memory foam occlusive element 24, thus having a diameter that is about equal to that of the shape memory foam occlusive element 24. In some cases, the outer ring 84 may be positioned within the shape memory foam occlusive element 24, and may not be visible, meaning that the outer ring 84 has a diameter that is less than a diameter of the shape memory foam occlusive element 24. While three struts 82 are shown, this is merely illustrative, as the reinforcing member 78 may include any number of struts 82. The outer ring 84 may be considered as an example of a foam integration structure that helps to anchor the reinforcing member 78 within the shape memory foam occlusive element 24. In some cases, one or more of the struts 82 may simply terminate, before reaching the outer ring 84. In some cases, the reinforcing member 78 may be adapted to collapse down to accommodate a compressed or crimped delivery configuration for the shape memory foam occlusive element 24, and may be adapted to automatically expand into an expanded configuration when the LAAC device 76 is delivered and is allowed to expand within the LAA 10.

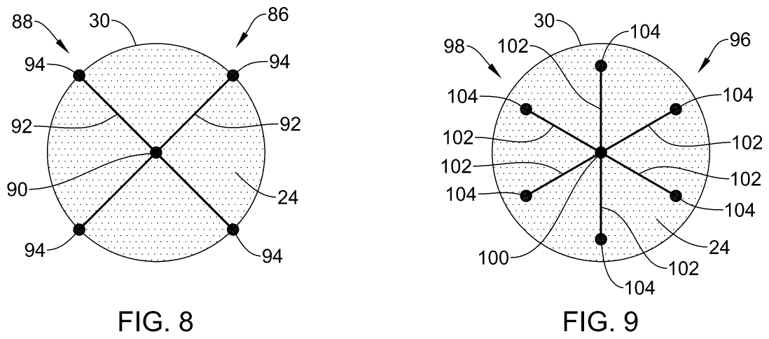

FIG. 8 is a schematic end view of an illustrative LAAC device 86 that combines the shape memory foam occlusive element 24 with a reinforcing member 88. The reinforcing member 88 may be considered as being an example of the reinforcing member 32 shown in FIG. 1. While a circular shape of the LAAC device 86 is shown, this is merely illustrative, as the LAAC device 86 may assume other shapes. As shown, the reinforcing member 88 includes a central point 90 that may or may not be visible along the proximal face 28. The reinforcing member 88 includes several struts 92 that extend radially outwardly from the central point 90, and extend axially into the shape memory foam occlusive element 24. In some cases, each of the struts 92 terminate in a ball 94. In some cases, the ball 94 for each strut 92 may be positioned at or near the periphery 30 of the shape memory foam occlusive element 24. In some cases, the ball 94 for at least some of the struts 92 may be positioned within the shape memory foam occlusive element 24, and may not be visible. While four struts 92 are shown, this is merely illustrative, as the reinforcing member 88 may include any number of struts 94. The balls 94 may be considered as examples of foam integration structures that help to anchor the reinforcing member 88 within the shape memory foam occlusive element 24. In some cases, one or more of the struts 92 may simply terminate, without a ball 94. In some cases, the reinforcing member 88 may be adapted to collapse down to accommodate a compressed or crimped delivery configuration for the shape memory foam occlusive element 24, and may be adapted to automatically expand into an expanded configuration when the LAAC device 86 is delivered and is allowed to expand within the LAA 10.

FIG. 9 is a schematic end view of an illustrative LAAC device 96 that combines the shape memory foam occlusive element 24 with a reinforcing member 98. The reinforcing member 98 may be considered as being an example of the reinforcing member 32 shown in FIG. 1. While a circular shape of the LAAC device 96 is shown, this is merely illustrative, as the LAAC device 96 may assume other shapes. As shown, the reinforcing member 98 includes a central point 100 that may or may not be visible along the proximal face 28. The reinforcing member 98 includes several struts 102 that extend radially outwardly from the central point 100, and extend axially into the shape memory foam occlusive element 24. In some cases, each of the struts 102 terminate in a ball 104. In some cases, the ball 104 for each strut 102 may be positioned at or near the periphery 30 of the shape memory foam occlusive element 24. In some cases, the ball 104 for at least some of the struts 102 may be positioned within the shape memory foam occlusive element 24, and may not be visible. While six struts 102 are shown, this is merely illustrative, as the reinforcing member 98 may include any number of struts 102. The balls 104 may be considered as examples of foam integration structures that help to anchor the reinforcing member 98 within the shape memory foam occlusive element 24. In some cases, one or more of the struts 102 may simply terminate, without a ball 104. In some cases, the reinforcing member 98 may be adapted to collapse down to accommodate a compressed or crimped delivery configuration for the shape memory foam occlusive element 24, and may be adapted to automatically expand into an expanded configuration when the LAAC device 96 is delivered and is allowed to expand within the LAA 10.

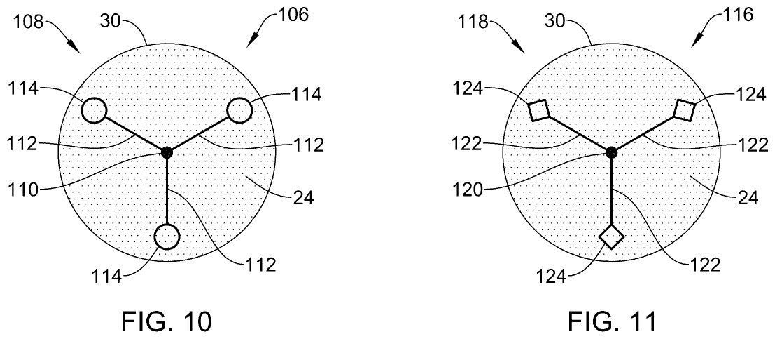

FIG. 10 is a schematic end view of an illustrative LAAC device 106 that combines the shape memory foam occlusive element 24 with a reinforcing member 108. The reinforcing member 108 may be considered as being an example of the reinforcing member 32 shown in FIG. 1. While a circular shape of the LAAC device 106 is shown, this is merely illustrative, as the LAAC device 106 may assume other shapes. As shown, the reinforcing member 108 includes a central point 110 that may or may not be visible along the proximal face 28. The reinforcing member 108 includes several struts 112 that extend radially outwardly from the central point 110, and extend axially into the shape memory foam occlusive element 24. In some cases, each of the struts 112 terminate in an open ring 114. In some cases, the open ring 114 for each strut 112 may be positioned at or near the periphery 30 of the shape memory foam occlusive element 24. In some cases, the open ring 114 for at least some of the struts 112 may be positioned within the shape memory foam occlusive element 24, and may not be visible. While three struts 112 are shown, this is merely illustrative, as the reinforcing member 108 may include any number of struts 112. The open rings 114 may be considered as examples of foam integration structures that help to anchor the reinforcing member 108 within the shape memory foam occlusive element 24. In some cases, one or more of the struts 112 may simply terminate, without an open ring 114. In some cases, the reinforcing member 108 may be adapted to collapse down to accommodate a compressed or crimped delivery configuration for the shape memory foam occlusive element 24, and may be adapted to automatically expand into an expanded configuration when the LAAC device 106 is delivered and is allowed to expand within the LAA 10.

FIG. 11 is a schematic end view of an illustrative LAAC device 116 that combines the shape memory foam occlusive element 24 with a reinforcing member 118. The reinforcing member 118 may be considered as being an example of the reinforcing member 32 shown in FIG. 1. While a circular shape of the LAAC device 116 is shown, this is merely illustrative, as the LAAC device 116 may assume other shapes. As shown, the reinforcing member 118 includes a central point 120 that may or may not be visible along the proximal face 28. The reinforcing member 118 includes several struts 122 that extend radially outwardly from the central point 120, and extend axially into the shape memory foam occlusive element 24. In some cases, each of the struts 122 terminate in an open diamond shape 124. In some cases, the open diamond shape 124 for each strut 122 may be positioned at or near the periphery 30 of the shape memory foam occlusive element 24. In some cases, the open diamond shape 124 for at least some of the struts 122 may be positioned within the shape memory foam occlusive element 24, and may not be visible. While three struts 122 are shown, this is merely illustrative, as the reinforcing member 118 may include any number of struts 122. The open diamond shapes 124 may be considered as examples of foam integration structures that help to anchor the reinforcing member 118 within the shape memory foam occlusive element 24. In some cases, one or more of the struts 122 may simply terminate, without an open diamond shape 124. In some cases, the reinforcing member 118 may be adapted to collapse down to accommodate a compressed or crimped delivery configuration for the shape memory foam occlusive element 24, and may be adapted to automatically expand into an expanded configuration when the LAAC device 116 is delivered and is allowed to expand within the LAA 10.

FIG. 12 is a schematic end view of an illustrative LAAC device 126 that combines the shape memory foam occlusive element 24 with a reinforcing member 128. The reinforcing member 128 may be considered as being an example of the reinforcing member 32 shown in FIG. 1. While a circular shape of the LAAC device 126 is shown, this is merely illustrative, as the LAAC device 126 may assume other shapes. As shown, the reinforcing member 128 includes a central point 130 that may or may not be visible along the proximal face 28. The reinforcing member 128 includes several struts 132 that extend radially outwardly from the central point 130, and extend axially into the shape memory foam occlusive element 24. In some cases, each of the struts 132 terminate in a bifurcated structure 134. In some cases, the bifurcated structure 134 for each strut 132 may be positioned at or near the periphery 30 of the shape memory foam occlusive element 24. In some cases, the bifurcated structure 134 for at least some of the struts 132 may be positioned within the shape memory foam occlusive element 24, and may not be visible. While three struts 132 are shown, this is merely illustrative, as the reinforcing member 128 may include any number of struts 132. The bifurcated structures 134 may be considered as examples of foam integration structures that help to anchor the reinforcing member 128 within the shape memory foam occlusive element 24. In some cases, one or more of the struts 132 may simply terminate, without a bifurcated structure 134. In some cases, the reinforcing member 128 may be adapted to collapse down to accommodate a compressed or crimped delivery configuration for the shape memory foam occlusive element 24, and may be adapted to automatically expand into an expanded configuration when the LAAC device 126 is delivered and is allowed to expand within the LAA 10.

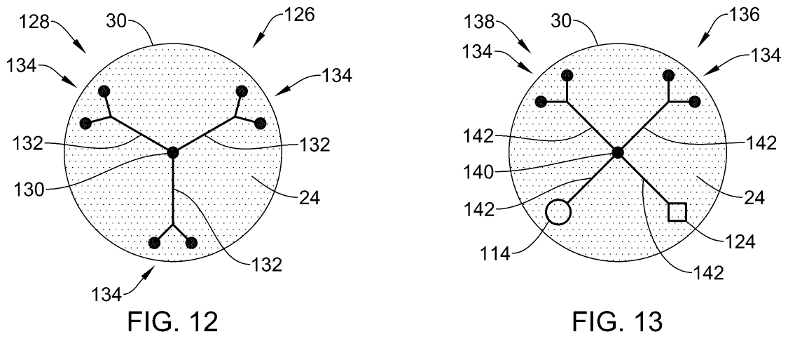

FIG. 13 is a schematic end view of an illustrative LAAC device 136 that combines the shape memory foam occlusive element 24 with a reinforcing member 138. The reinforcing member 138 may be considered as being an example of the reinforcing member 32 shown in FIG. 1. While a circular shape of the LAAC device 136 is shown, this is merely illustrative, as the LAAC device 136 may assume other shapes. As shown, the reinforcing member 138 includes a central point 140 that may or may not be visible along the proximal face 28. The reinforcing member 138 includes several struts 142 that extend radially outwardly from the central point 140, and extend axially into the shape memory foam occlusive element 24. In some cases, each of the struts 142 terminate in a foam integration structure. As shown, two of the struts 142 terminate in the bifurcated structure 134, one of the struts 142 terminate in the open ring 114 and one of the struts 142 terminates in the open diamond shape 124. In some cases, the foam integration structure for each strut 142 may be positioned at or near the periphery 30 of the shape memory foam occlusive element 24. In some cases, the foam integration structure for at least some of the struts 142 may be positioned within the shape memory foam occlusive element 24, and may not be visible. While four struts 142 are shown, this is merely illustrative, as the reinforcing member 138 may include any number of struts 142. In some cases, one or more of the struts 142 may simply terminate, without a foam integration structure. In some cases, the reinforcing member 138 may be adapted to collapse down to accommodate a compressed or crimped delivery configuration for the shape memory foam occlusive element 24, and may be adapted to automatically expand into an expanded configuration when the LAAC device 136 is delivered and is allowed to expand within the LAA 10.

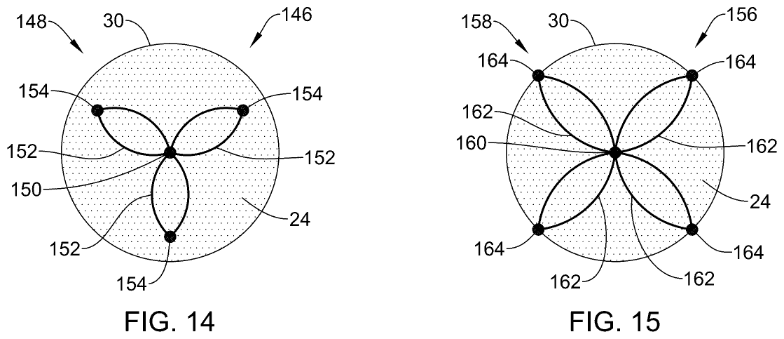

FIG. 14 is a schematic end view of an illustrative LAAC device 146 that combines the shape memory foam occlusive element 24 with a reinforcing member 148. The reinforcing member 148 may be considered as being an example of the reinforcing member 32 shown in FIG. 1. While a circular shape of the LAAC device 146 is shown, this is merely illustrative, as the LAAC device 146 may assume other shapes. As shown, the reinforcing member 148 includes a central point 150 that may or may not be visible along the proximal face 28. The reinforcing member 148 includes several flower petal-shaped features 152 that extend radially outwardly from the central point 150, and extend axially into the shape memory foam occlusive element 24. In some cases, each of the flower petal-shaped features 152 terminate in a ball 154. In some cases, the balls 154 for each flower petal-shaped feature 152 may be positioned at or near the periphery 30 of the shape memory foam occlusive element 24. In some cases, the balls 154 for at least some of the flower petal-shaped features 152 may be positioned within the shape memory foam occlusive element 24, and may not be visible. While three flower petal-shaped features 152 are shown, this is merely illustrative, as the reinforcing member 148 may include any number of flower petal-shaped features 152. The balls 154 may be considered as examples of foam integration structures that help to anchor the reinforcing member 148 within the shape memory foam occlusive element 24. In some cases, one or more of the flower petal-shaped features 152 may simply terminate, without a ball 154. In some cases, the reinforcing member 148 may be adapted to collapse down to accommodate a compressed or crimped delivery configuration for the shape memory foam occlusive element 24, and may be adapted to automatically expand into an expanded configuration when the LAAC device 146 is delivered and is allowed to expand within the LAA 10.

FIG. 15 is a schematic end view of an illustrative LAAC device 156 that combines the shape memory foam occlusive element 24 with a reinforcing member 158. The reinforcing member 158 may be considered as being an example of the reinforcing member 32 shown in FIG. 1. While a circular shape of the LAAC device 156 is shown, this is merely illustrative, as the LAAC device 156 may assume other shapes. As shown, the reinforcing member 158 includes a central point 160 that may or may not be visible along the proximal face 28. The reinforcing member 158 includes several flower petal-shaped features 162 that extend radially outwardly from the central point 160, and extend axially into the shape memory foam occlusive element 24. In some cases, each of the flower petal-shaped features 162 terminate in a ball 164. In some cases, the balls 164 for each flower petal-shaped feature 162 may be positioned at or near the periphery 30 of the shape memory foam occlusive element 24. In some cases, the balls 164 for at least some of the flower petal-shaped features 162 may be positioned within the shape memory foam occlusive element 24, and may not be visible. While four flower petal-shaped features 162 are shown, this is merely illustrative, as the reinforcing member 158 may include any number of flower petal-shaped features 162. The balls 154 may be considered as examples of foam integration structures that help to anchor the reinforcing member 158 within the shape memory foam occlusive element 24. In some cases, one or more of the flower petal-shaped features 162 may simply terminate, without a ball 164. In some cases, the reinforcing member 158 may be adapted to collapse down to accommodate a compressed or crimped delivery configuration for the shape memory foam occlusive element 24, and may be adapted to automatically expand into an expanded configuration when the LAAC device 156 is delivered and is allowed to expand within the LAA 10.

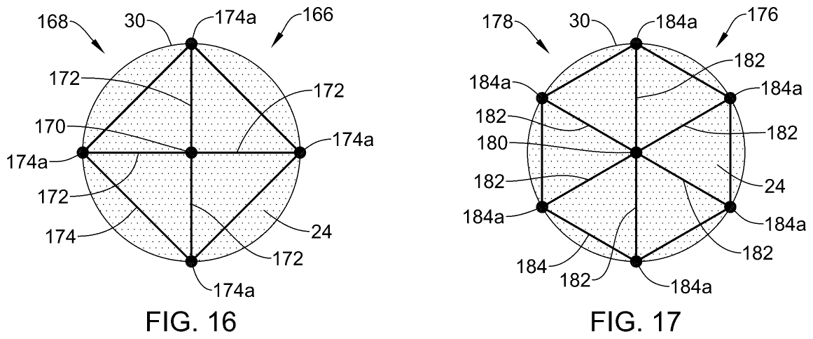

FIG. 16 is a schematic end view of an illustrative LAAC device 166 that combines the shape memory foam occlusive element 24 with a reinforcing member 168. The reinforcing member 168 may be considered as being an example of the reinforcing member 32 shown in FIG. 1. While a circular shape of the LAAC device 166 is shown, this is merely illustrative, as the LAAC device 166 may assume other shapes. As shown, the reinforcing member 168 includes a central point 170 that may or may not be visible along the proximal face 28. The reinforcing member 168 includes several struts 172 that extend radially outwardly from the central point 170, and extend axially into the shape memory foam occlusive element 24. In some cases, as shown, each of the struts 172 terminate at an outer square 174. In some cases, the outer square 174 may be positioned at or near the periphery 30 of the shape memory foam occlusive element 24. In some cases, the outer square 174 may be positioned within the shape memory foam occlusive element 24, and may not be visible. While four struts 172 are shown, this is merely illustrative, as the reinforcing member 168 may include any number of struts 172. The outer square 174 may be considered as an example of a foam integration structure that helps to anchor the reinforcing member 168 within the shape memory foam occlusive element 24. In some cases, one or more of the struts 172 may simply terminate, before reaching the outer square 174. In some cases, each of the struts 172 may be joined to the outer square 174 via a ball joint 174a. In some cases, the reinforcing member 168 may be adapted to collapse down to accommodate a compressed or crimped delivery configuration for the shape memory foam occlusive element 24, and may be adapted to automatically expand into an expanded configuration when the LAAC device 166 is delivered and is allowed to expand within the LAA 10.

FIG. 17 is a schematic end view of an illustrative LAAC device 176 that combines the shape memory foam occlusive element 24 with a reinforcing member 178. The reinforcing member 178 may be considered as being an example of the reinforcing member 32 shown in FIG. 1. While a circular shape of the LAAC device 176 is shown, this is merely illustrative, as the LAAC device 176 may assume other shapes. As shown, the reinforcing member 178 includes a central point 180 that may or may not be visible along the proximal face 28. The reinforcing member 178 includes several struts 182 that extend radially outwardly from the central point 180, and extend axially into the shape memory foam occlusive element 24. In some cases, as shown, each of the struts 182 terminate at an outer hexagon 184. In some cases, the outer hexagon 184 may be positioned at or near the periphery 30 of the shape memory foam occlusive element 24. In some cases, the outer hexagon 184 may be positioned within the shape memory foam occlusive element 24, and may not be visible. While six struts 182 are shown, this is merely illustrative, as the reinforcing member 178 may include any number of struts 182. The outer hexagon 184 may be considered as an example of a foam integration structure that helps to anchor the reinforcing member 178 within the shape memory foam occlusive element 24. In some cases, one or more of the struts 182 may simply terminate, before reaching the outer hexagon 184. In some cases, each of the struts 182 may be joined to the outer hexagon 184 via a ball joint 184a. In some cases, the reinforcing member 178 may be adapted to collapse down to accommodate a compressed or crimped delivery configuration for the shape memory foam occlusive element 24, and may be adapted to automatically expand into an expanded configuration when the LAAC device 176 is delivered and is allowed to expand within the LAA 10.

The reinforcing members 32, 42, 48, 58, 68, 78, 88, 98, 108, 118, 128, 138, 148, 148, 168 and 178, as well as components thereof may be made of any suitable material. The reinforcing members 32, 42, 48, 58, 68, 78, 88, 98, 108, 118, 128, 138, 148, 148, 168 and 178 may be made of a shape memory polymer or shape memory foam that differs from a foam used to create the shape memory foam occlusive element 24. The reinforcing members 32, 42, 48, 58, 68, 78, 88, 98, 108, 118, 128, 138, 148, 148, 168 and 178 may be made of any suitable metallic material. Individual components of the reinforcing members 32, 42, 48, 58, 68, 78, 88, 98, 108, 118, 128, 138, 148, 148, 168 and 178 may be adhesively secured together or welded together, for example.

The expandable foam may include any suitable material, such as a suitable polymeric material, that is capable of transitioning from an initial configuration to an expanded configuration upon being subjected to a specific temperature or temperature range and/or exposure to moisture, and provide a suitable density in the expanded configuration for use inside of the left atrial appendage to provide an occlusive benefit without negatively impacting surrounding anatomy. In some instances, the expandable foam may be a shape memory foam. Suitable transition temperatures may be, for example, at or below about 37° C. (98.6° F.), which allows the shape memory foam to assume an initial configuration prior to and during delivery through a delivery catheter or other delivery device, and an expanded configuration for occlusion after delivery and release within the left atrial appendage, allowing the shape memory foam to be exposed to body temperature blood within the left atrial appendage. A suitable density of the shape memory foam in the expanded configuration is a density that allows the expanded configuration to be pliable and compliant and substantially conform to the left atrial appendage anatomy to create a seal to protect against the formation and escape of blood clots while having sufficient radial force to seal the left atrial appendage but not damage or impact surrounding anatomy. In some instances, the density of the shape memory foam in the expanded configuration will be from about 10 kg/m³ (about 0.62 lb/ft³) to about 1000 kg/m³ (about 62.311 b/ft³), including from about 10 kg/m³ to about 500 kg/m³ (about 31.2 lb/ft³) including from about 10 kg/m³ to about 200 kg/m³ (about 12.5 lb/ft³), including from about 20 kg/m³ to about 100 kg/m³ (about 6.2 lb/ft³).

Generally, the material for constructing the shape memory foam is a polymeric material that is both biocompatible and substantially biostable. In some instances, biocompatibility will include meeting or surpassing the requirements of established standards for implant materials defined in ISO 10993 and USP Class VI. Substantially biostable materials include those materials that do not resorb over the intended lifetime of the medical device (such as five years, or ten years, or longer), as well as those materials that resorb slowly such that void volume is replaced by a stable tissue-like material over a period of a few months to a year.

In some instances, the shape memory foam may include a natural and/or synthetic material. Suitable natural materials may include, for example, extracellular matrix (ECM) biopolymers such as collagen, fibronectin, hyaluronic acid and elastin, non-ECM biomaterials such as cross-linked albumin, fibrin, and inorganic bioceramics such as hydroxyapatite and tricalcium phosphate. Suitable synthetic materials may include, for example, biostable polymers such as saturated and unsaturated polyolefins including polyethylene, polyacrylics, polyacrylates, polymethacrylates, polyamides, polyimides, polyurethanes, polyureas, polyvinyl aromatics such as polystyrene, polyisobutylene copolymers and isobutylene-styrene block copolymers such as styrene-isobutylene-styrene tert-block copolymers (SIBS), polyvinylpyrolidone, polyvinyl alcohols, copolymers of vinyl monomers such as ethylene vinyl acetate (EVA), polyvinyl ethers, polyesters including polyethylene terephthalate, polyacrylamides, polyethers such as polyethylene glycol, polytetrahydrofuran and polyether sulfone, polycarbonates, silicones such as siloxane polymers, and fluoropolymers such as polyvinylidene fluoride, and mixtures and copolymers of the above.

In some instances, the shape memory foam may include a bioresorbable material such that resorption results in the formation of a biostable tissue matrix. Synthetic bioresorbable polymers may, for example, be selected from the following: (a) polyester homopolymers and copolymers such as polyglycolide (PGA; polyglycolic acid), polylactide (PLA; polylactic acid) including poly-L-lactide, poly-D-lactide and poly-D,L-lactide, poly(beta-hydroxybutyrate), polygluconate including poly-D-gluconate, poly-L-gluconate, poly-D,L-gluconate, poly(epsilon-caprolactone), poly(delta-valerolactone), poly(p-dioxanone), poly(lactide-co-glycolide) (PLGA), poly(lactide-codelta-valerolactone), poly(lactide-co-epsilon-caprolactone), poly(lactide-co-beta-malic acid), poly(beta-hydroxybutyrate-co-beta hydroxyvalerate), poly[1,3bis(p-carboxyphenoxy)propane-co-sebacic acid], and poly(sebacic acid-co-fumaric acid); (b) polycarbonate homopolymers and copolymers such as poly(trimethylene carbonate), poly(lactide-co-trimethylene carbonate) and poly(glycolide-co-trimethylene carbonate); (c) poly(ortho ester homopolymers and copolymers such as those synthesized by copolymerzation of various diketene acetals and diols; (d) polyanhydride homopolymers and copolymers such as poly(adipic anhydride), poly(suberic anhydride), poly (sebacic anhydride), poly(dodecanedioic anhydride), poly(maleic anhydride), poly[1,3-bis-(p-carboxyphenoxy)methane anhydride], and poly[alpha,omega-bis(p-carboxyphenoxy)alkane anhydride] such as poly[1,3-bis(p-carboxyphenoxy)propane anhydride] and poly[1,3-bis(p-carboxyphenoxy)hexane anhydride]; (e) polyphosphazenes such as aminated and alkoxy substituted polyphosphazenes; and (f) amino-acid-based polymers including tyrosine-based polymers such as tyrosine-based polyacrylates (e.g., copolymers of a diphenol and a diacid linked by ester bonds, with diphenols selected, for example, from ethyl, butyl, hexyl, octyl, and benzyl esters of desaminotyrosyl-tyrosine and diacids selected, for example, from succinic, glutaric, adipic, suberic, and sebacic acid), tyrosine-based polycarbonates (e.g., copolymers formed by the condensation polymerization of phosgene and a diphenol selected, for example, from ethyl, butyl, hexyl, octyl, and benzyl esters of desaminotyrosyl-tyrosine, tyrosine-based iminocarbonates, and tyrosine-, leucine- and lysine-based polyester-amides; specific examples of tyrosine-based polymers further include polymers that are comprised of a combination of desaminotyrosyl tyrosine hexyl ester, desaminotyrosyl tyrosine, and various di-acids, for example, succinic acid and adipic acid. Suitable materials include cross-linked polycarbonates and crosslinked polyethylene glycols.

In some instances, the shape memory foam may include thermoset polyurethanes that include oxidatively susceptible linkages in the soft segment, including but not limited to tertiary amines and polyethers. The shape memory foam may optionally include hydrolytically degradable soft segment components such as polycaprolactone, esters, and others.

A shape memory foam may have a thermal transition point (transition temperature) below which residual stress is maintained without a loading constraint. The thermal activation (which causes the shape memory) may be achieved with the desired material going through a semi-crystalline melt point or glass transition temperature between the first configuration and the expanded configuration. Several suitable thermal activation processes are known in the art and useful herein. In an example, the temperature activated memory shape foam may be formed for use as a medical device by first shaping a shape memory foam formed from a suitable material into its final expanded configuration; that is, the configuration that the shape memory shape foam will achieve once inserted into the left atrial appendage to provide the desired occlusive benefit. In this expanded configuration, the shape memory foam may generally have a diameter that will range from about 10 millimeters (about 0.39 inches) to about 50 millimeters (about 1.97 inches) and a length that will range from about 1 centimeter (about 0.39 inches) to about 5 centimeters (about 1.97 inches), although other diameters and lengths are within the scope of the present disclosure. Once this has been done, the foam may be heated above the transition temperature of the material; that is, the temperature at which a desired expansion will occur. Once the desired transition temperature has been achieved, the shape memory foam is held at a constant temperature and is re-shaped into an initial (unexpanded) configuration. This re-shaping is suitably done in a properly sized molding element and may be any suitable shape.

After insertion into the molding element, the temperature is reduced to a temperature below the transition temperature to set the new shape; for example, the temperature may be reduced to room temperature to set the new shape. Once this has been completed, the shape memory foam will remain in its first configuration until it is subjected to a temperature at or above the transition temperature, at which time it will expand into its expanded, or remembered, configuration. In some instances, exposure to water within the blood changes the glass transition temperature of the foam. As an example, the shape memory foam may have a dry T_{g} (glass transition temperature) that is above body temperature, and may have a wet T_{g}, after exposure to water, that is lower than body temperature.

In some cases, the shape memory polymers may include non-foamed versions of the polymers described herein with respect to making the expandable foams such as shape memory foams. Example of bio-compatible shape memory polymers include polymers made from poly(s-caprolactone) (PCL), polyurethane (PU), poly (D, L-lactide) (PDLLA), PVA, ethylene vinyl acetate copolymer, (EVA) polymer blend, polymer composites, crosslinked polymers and supramolecular networks, among others. In some instances, shape memory polymers that may be used in creating the anchors described herein may include polyurethane, for example.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22@, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2@), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHY NOX®, and the like); platinum enriched stainless steel; titanium; combinations thereof; and the like; or any other suitable material.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, the devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHY NOX®, and the like), nickel-cobalt-chromium-molybdenum alloys (e.g., UNS: R30035 such as MP35-N® and the like), nitinol, and the like, and others.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A left atrial appendage closure (LAAC) device adapted for occluding a patient's left atrial appendage (LAA), the LAAC device comprising:
a shape memory foam occlusive element adapted to expand from a crimped configuration for delivery to an expanded configuration after delivery, the shape memory foam occlusive element including in its expanded configuration a distal face, a proximal face and an intervening periphery; and
a reinforcing member disposed relative to the shape memory foam occlusive element, the reinforcing member adapted to limit deformation of the shape memory foam occlusive element after the shape memory foam occlusive element has expanded into the expanded configuration.

2. The LAAC device of claim 1, wherein the reinforcing member extends around at least a portion of the periphery of the shape memory foam occlusive element.

3. The LAAC device of claim 1, wherein the reinforcing member extends across at least a portion of the distal face of the shape memory foam occlusive element.

4. The LAAC device of claim 1, wherein the reinforcing member extends across at least a portion of the proximal face of the shape memory foam occlusive element.

5. The LAAC device of claim 1, wherein at least a portion of the reinforcing member extends through the shape memory foam occlusive element.

6. The LAAC device of claim 1, wherein the reinforcing member is adapted to expand with the shape memory foam occlusive element when the shape memory foam occlusive element expands after deployment.

7. The LAAC device of claim 1, wherein the reinforcing member comprises a second foam different from a first foam forming the shape memory foam occlusive element.

8. The LAAC device of claim 1, wherein the reinforcing member comprises a polymeric structure.

9. The LAAC device of claim 1, wherein the reinforcing member comprises a metallic structure.

10. A left atrial appendage closure (LAAC) device adapted for occluding a patient's left atrial appendage (LA A), the LAA including a proximal mouth, the LAAC device comprising:
a deformable device body adapted to expand and span the proximal mouth of the LAA; and
a control structure adapted to limit deformation of the deformable device body once expanded.

11. The LAAC device of claim 10, wherein the deformable device body comprises a shape memory foam.

12. The LAAC device of claim 10, wherein the control structure extends around at least a portion of a periphery of the deformable device body.

13. The LAAC device of claim 10, wherein the control structure extends across at least a portion of a distal side of the deformable device body.

14. The LAAC device of claim 10, wherein the reinforcing member extends across at least a portion of a proximal side of the deformable device body.

15. The LAAC device of claim 10, wherein at least a portion of the reinforcing member extends through the deformable device body.

16. A left atrial appendage closure (LAAC) device adapted for occluding a patient's left atrial appendage (LAA), the LAAC device comprising:
a shape memory foam occlusive element adapted to expand from a delivery configuration to a deployment configuration; and
a reinforcing member disposed relative to the shape memory foam occlusive element, the reinforcing member including a central point and a plurality of struts emanating outwardly from the central point.

17. The LAAC device of claim 16, wherein the reinforcing member is adapted to limit deformation of the shape memory foam occlusive element after the shape memory foam occlusive element has expanded.

18. The LAAC device of claim 16, wherein the reinforcing member further comprises an annular ring extending around and connected to at least some of the plurality of struts.

19. The LAAC device of claim 18, wherein the annular ring has a diameter that is equal to or less than an overall diameter of the shape memory foam occlusive element.

20. The LAAC device of claim 16, wherein the reinforcing member further comprises one or more foam integration structures emanating from at least some of the plurality of struts.
